(19) United States
(12) Patent Application Publication
(10) Pub. No.: US 2026/0263239 A1
(43) Pub. Date: Sep. 10, 2026

(54) ORTHOPEDIC AND SPINAL SURGERY SYSTEMS AND DEVICES FOR PREPATORY MEASUREMENT

(71) Applicant: OrthoIQ, LLC, Claremont, CA (US)

(72) Inventors: Philip Ormond Merritt, La Canada, CA (US); Alan Aaron Davidner, Claremont, CA (US)

(21) Appl. No.: 19/546,879

(22) Filed: Feb. 23, 2026

Related U.S. Application Data
(60) Provisional application No. 63/762,383, filed on Feb. 24, 2025.

(51) Int. Cl. A61F 2/46; A61B 90/00

(52) U.S. Cl. CPC A61F 2/4657; A61B 90/361; A61B 2090/373; A61B 2090/3937; A61F 2002/4668

(57) ABSTRACT

An orthopedic surgical system may include a goniometer including a flexible sensor having a first end and a second end. The first end is coupled to a reference structure, and the flexible sensor generates measurement phenomenon correlated to a relative position of the first end and the second end. The measurement phenomenon is selected from the group consisting of an electrical resistance, a capacitance, a current, a pressure on a piezoelectric substrate, a magnetic field, a magnetic angle, or a mechanical force. A system may include a processing unit communicatively coupled to the goniometer, such that cause the processing unit performs a method including receiving a first signal indicative of a first value for the measurement phenomenon, correlating the first signal with a first angle between the first end and the second end of the flexible sensor, and displaying a value of the first angle.

### CROSS-REFERENCE TO RELATED APPLICATIONS

This application claims the priority benefit of U.S. Provisional Patent Application Ser. No. 63/762,383, filed Feb. 24, 2025, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates to the field of orthopedic and spinal surgery (e.g., reconstructive and repair surgery of the hip, knee, shoulder, lumbar, thoracic and cervical regions, and more specifically, to the preparation of the bone for the placement of implant components, fixation and repair devices.) Described herein are systems, components, and methods for easily, safely and effectively preparing and/or repairing a bone for implantation or devices into the bone for hip, knee and shoulder arthroplasty, or lumber, thoracic and cervical fusion.

### BACKGROUND

The frequency of total hip arthroplasty procedures is expected to grow to 635,000 procedures by the year 2030. The frequency of total knee and partial knee arthroplasty is expected to exceed 1.2 million procedures by the year 2030. The frequency of standard total shoulder arthroplasty and reverse shoulder arthroplasty procedures is expected to grow to 400,000 procedures by the year 2030. The frequency of spinal fusion procedures is expected to conservatively grow to 650,000 procedures by 2030. These procedures are effective in relieving pain and improving function. These procedures allow patients to return to normal function status in a relatively short period of time. The more accurately the implants and repairs are performed, the more rapidly a patient can return to function. Accurate implantation and repair have been shown to improve rehabilitation, decrease complications, decrease hospital stays and significantly reduce costs. When complications occur, they may require revision surgery which can increase the cost of treatment by as much as $150,000 or more. The ability to decrease the complication rate can significantly increase the success of surgery and return the patient to a functional status quickly.

### SUMMARY

In some embodiments, the techniques described herein relate to an angular and distance measurement system including a flexible single, biaxial or triaxial angular sensor, and a camera that employs astronomical image processing or an equivalent software capability. The flexible angular sensor may function through changes in resistance, capacitance, current generated by a piezoelectric substrate or a magnetic angle position sensor, in some embodiments. The distance measurement system may employ a camera and, in some embodiments, an ultrasonic height measuring system that is attached to an operating room overhead light handle. In some embodiments, the flexible angular sensor (e.g., goniometer) may be used separately from the distance measurement system. In some embodiments, the distance measurement system may be used separately from the flexible angular sensor.

In some embodiments, the techniques described herein relate to a flexible angular sensor for orthopedic and spinal fusion surgery. The flexible angular sensor may have a first side functioning as a reference plane and a second side for determining angle changes. For example, a flexible angular sensor (e.g., a first sensor) may be attached on one end (a first end) to a reference plane or structure including, but not limited to, a coronal (horizontal) reference plane pelvis, arm, leg, mayo stand, L bracket affixed to a gurney rail, or at a fixture oriented in the sagittal plane (vertical mid plane dividing the patient). The opposite end of the sensor (a second end) or a second sensor in communication with the first sensor establishes an angle relative to the reference first sensor end by a change in resistance, capacitance, pressure on a piezoelectric substrate, or change in magnetic field with a change in position or angle. Thus, an angle and a change in the angle creates a signal that is sent to an attached display (e.g., an LED or LCD display) providing useful orthopedic and spinal surgical angle information. The angle information may include, but is not limited to, tibial posterior angle, varus, valgus, inclination, anteversion, reverse and total shoulder angle, humerus cut angles as well as pilot hole, bone screw tapping device, bone screw, spinal fixation device and/or spinal fixation instrument angle.

Additionally, in some embodiments, the signal that is sent to the display can also be sent wirelessly to a handheld device, computer, laptop, navigation device, virtual or augmented reality device or robot for inclusion in a patient record.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates an embodiment of a flexible orthopedic single or multiple axis surgical goniometer(s) employed as at least part of a measurement system in partial or total knee arthroplasty to establish a posterior angle for a tibial saw cut along with both a valgus and varus angle determination.

FIG. 1B illustrates an embodiment of the assembled components in the single or multiple axis flexible knee surgical goniometer(s) as at least part of a measurement system.

FIG. 2A illustrates an embodiment of an orthopedic single or multiple axis flexible surgical goniometer(s) employed as at least part of a measurement system in hip arthroplasty to establish an inclination and anteversion angle for the installation of an acetabular cup with an acetabular handle.

FIG. 2B illustrates an embodiment of the assembled components of an orthopedic single or multiple axis flexible hip surgical goniometer(s) as at least part of a measurement system.

FIG. 3A illustrates an embodiment of an orthopedic hip arthroplasty distance measurer as at least part of a measurement system that employs the principles of advanced image processing to determine offset and leg length during hip arthroplasty.

FIG. 3B illustrates a pelvic bone with marker dots for reference.

FIGS. 4A and 4B illustrate embodiments of an orthopedic single or multiple axis flexible goniometer(s) employed as at least part of a measurement system in reverse shoulder arthroplasty to establish the RSA angle or inferior glenoid angle (12 to 20 degrees) for the installation of a ball head and the humerus cut angle in two dimensions for the installation of a socket.

FIG. 4C illustrates an embodiment of the assembled components of an orthopedic single or multiple axis flexible reverse shoulder surgical goniometer(s) as at least part of a measurement system.

FIG. 5A illustrates an embodiment of a spinal fusion single or multiple axis flexible goniometer(s) employed as at least part of a measurement system in lumbar, thoracic and cervical fusion to establish proper alignment of a pedicle screw(s) with rods or cages.

FIG. 5B illustrates an embodiment of the assembled components in the single or multiple axis flexible spinal fusion goniometer(s) as at least part of a measurement system.

FIG. 6 is a block diagram illustrating the installation and operation of the flexible goniometer(s) as at least part of a measurement system.

FIG. 7 is a block diagram illustrating the installation and operation of a distance measurer as at least part of a measurement system.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

The goal with orthopedic arthroplasty surgery or spinal fusion surgery is the accurate implantation of the implantable components or repair of the musculoskeletal anatomy. This includes minimizing the time of surgery which decreases the time the wound is open, thus decreasing the infection rate and the overall complication rate including anesthetic complications. The existing computer-assisted, robotic, virtual reality and accelerometer/gyroscope-based systems are not only expensive, adding significant cost to the procedure, but are also time consuming and can add ten minutes to twice the usual time to perform surgery. For example, the cost of these systems may be as high as $1.2 million and increase the cost of each surgery by $1,000 to $5,000.

Of course, hip, knee, shoulder replacement surgery and spine repair surgery may also be performed without robotic, accelerometer/gyroscopes, virtual reality and computer assisted devices. In some cases, the added complexity of these devices does not necessarily translate to better patient outcomes compared to conventional surgery. In fact, many spinal fusion surgeries are performed without assistive guidance technology; however, more power assisted devices, robots, and computer assisted devices are used in spine fusion surgeries.

There is a cost of technology that translates to increased time and cost of the surgical procedure. This technology can increase the complexity of the surgery and uses additional time to calibrate and place instruments. This often includes surgical trauma to drill holes into the bone to anchor fixation pins in order to attach infrared trackers or fixtures. This leaves significant voids in the bones and increases the risk of fracture. Moreover, the voids act as stress risers and may temporarily weaken the bones. These flags and fixtures translate to increased time for calibration of instrumentation, time to collect the information and time to process the information. Often the surgeon is beset with overwhelming data that is not useful for the surgery being performed. This added information, therefore, can be distracting and lead to a loss of focus. Further, these technologies also have steep learning curves requiring many hours of training.

In a retrospective study of spinal fusion surgery by the NIH, there was a 42.1% angle mismatch between the pedicle screw anchoring system and the rods. The revision rate was 11.8%. The interface angle between the pedicle screw head “tulip” and the rod should be 90 degrees in order to avoid loosening or generating additional mechanical stress. This is especially important because the spinal column can see more than twice the force of lower body joints.

There can also be a mismatch between the pedicle screw pilot holes and the tapping element. This can result in damage to the neuro-vascular bed.

Orthopedic and spine surgeons struggle with the alignment process during the installation of implants or implements into bone (e.g., the femur, pelvis, tibia, humerus, and spinal column). A change in installation angle or linear measurement can result in misalignment in a joint or anatomical feature. Relying on a portable CT (O) scanner or a freehand approach may even compound the misalignment.

The systems and methods described herein solve the above problems by providing the surgeon with a way to control the installation angle or anatomical measurement with simple, accurate, and easy to use device(s). Described herein are applications of devices that either employ changes in capacitance, resistance, piezoelectric voltage, or magnetics as a direct correlation for angle measurement. Further, by counting the number of pixels in a camera shot between two anatomical points, accurate anatomical measurements can also be achieved.

The systems described herein may be sterile, single use, and/or disposable. Alternatively, they may be reusable such that they can be sterilized by ethylene oxide or gamma ray sterilization. They also can be designed to be reusable and autoclaved or cleaned in the hospital.

Another use case for such technology may be animals. For example, large dogs such as Great Danes, Saint Bernards, Labrador Retrievers, Mastiffs and German Shepherds can suffer from hip dysplasia, a genetic disease, and congenital and acquired spinal deformities, as can small and miniature horses. Implants and internal fixation devices used in these cases are often small, so the alignment can be more critical to a successful outcome.

As part of a measurement system, flexible goniometers may be used, as described herein, to measure a change in angle in at least one axis during a surgical procedure, in some embodiments. For example, a flexible 1-axis goniometer may measure rotation about the x-axis (e.g., tilt axis). For example, a flexible 2-axis goniometer may measure rotation about the x-axis (e.g., tilt axis) and rotation about the y-axis (e.g., pitch axis). In some embodiments, the flexible goniometer may additionally measure rotation about the z-axis (e.g., yaw axis). To measure changes in the one or more angles, the goniometer may be secured on a first end to a reference structure and to a surgical instrument on a second end, in some embodiments. The first and second end of the goniometer are in communication with each other and/or generate a measurement signal correlating to the relative position of the first and second end, in some embodiments, such that the measurement signal can be correlated to an angle in at least two axes. For example, the relative position of the first and second end may generate an electrical capacitance having a value that may be correlated with an angle between the first and second end. In some embodiments, the goniometer may additionally, or alternatively, use a change in resistance, current, pressure on a piezoelectric substrate, magnetic field, magnetic angle, or mechanical force (e.g., a force sensor) to measure an angle change. In some embodiments, the curvature or path of the flexible goniometer does not affect angle measurements, but may impact the relative position of the first and second end. In some embodiments, the measurable signals and their correlation to angles may be performed by a processor (e.g., digital signal processor, field programmable gate array, graphics processing unit, central processing unit, microcontroller, etc.) of a computing device in communication (e.g., wirelessly, wired connection, etc.) with the flexible goniometer. The computing device may be a mobile computing device, stationary computing device (e.g., desktop or workstation), a server, a virtual/augmented/mixed reality headset, or a microcontroller with a display attached to at least a portion of the measurement system. Optionally, A value of the one or more measured angles may be displayed on a display in communication with the goniometer in some embodiments.

In some embodiments, a surgical measurement system includes a flexible goniometer as described herein in combination with a distance measurer. The distance measurer may include a camera, and markers (e.g., dots, bone screws with caps, etc.) may be affixed to a patient's anatomy of interest during an operation, in some embodiments. A processor of the system in communication with the camera may process images of the markers captured by the camera to calculate an approximate center of the markers and a distance between at least two of the markers. Optionally, a display in communication with the system may display the distance between at least two of the markers.

As described herein, the surgical measurement system of the goniometer as described herein with or without the distance measurer provides technical solutions to users in the form of the generation and presentation of reliable and accurate operation parameter measurements without the use of expensive robotic technologies, time consuming technologies, like MRI, or accelerometer/gyroscope-technologies.

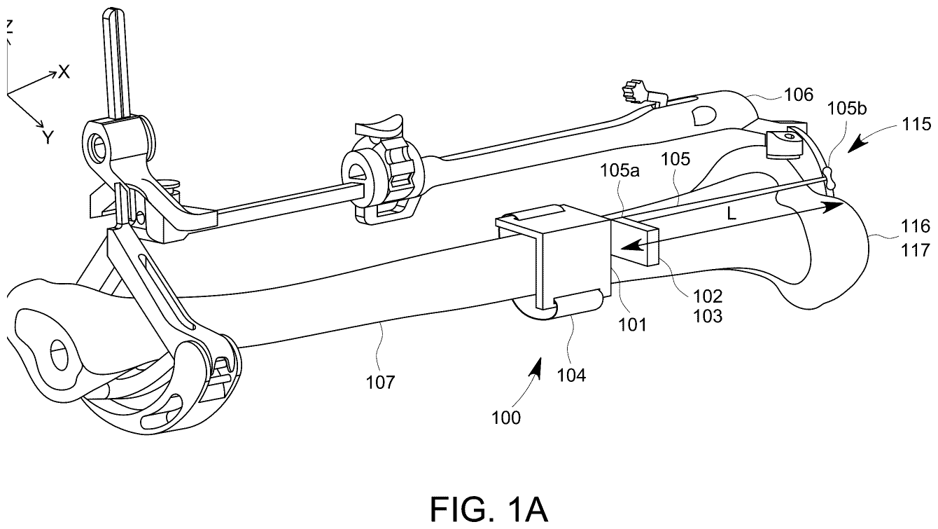

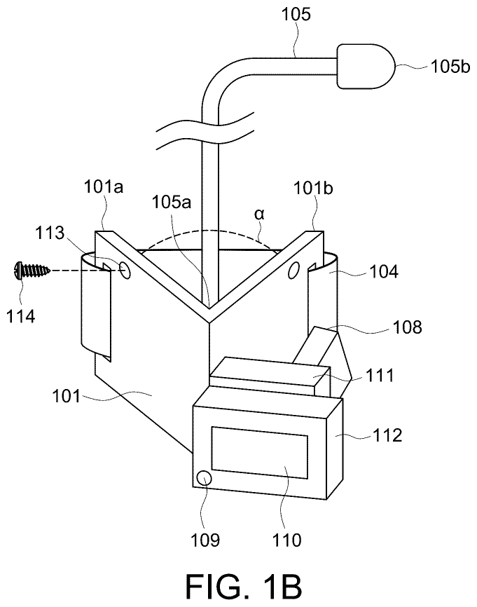

Now referring to FIGS. 1A-1B, in some embodiments a measurement system 100 is shown with a mounting platform 101, display 102, electronics 103, strap 104, and flexible 2-axisgoniometer 105 having a first end 105a and a second end 105b. In some embodiments, the second end 105b may be considered at the end of an extender of the goniometer 105. In FIG. 1A, a tibial cutting fixture 106 and tibia 107 are shown for reference. In some embodiments, the goniometer 105 may have a square-shaped cross-section, but other profiles and shapes may be employed (e.g., circular cross-section, triangular cross-section, polygonal cross-section, etc.).

As shown in FIG. 1B, internal components of the measurement system 100, in some embodiments, may include a plurality of components include, but not limited to, a battery 108, an on-off tare button 109, a display 110 (e.g., an LED or LCD display) with an internal driver (not shown), a printed circuit board 111, and an electronic enclosure or housing 112. In some embodiments, an angle α formed between a first plate 101a and a second plate 101b of the mounting platform 101 may be about 75 degrees to about 125 degrees, about 85 degrees to about 115 degrees, or about 90 degrees to about 110 degrees. Optional holes 113 in the mounting platform 101, in some embodiments, provide the surgeon with a way to choose from a strap 104 or a bone screw 114 as a means to secure the inferior end (e.g., first end 105a) of the flexible goniometer 105.

In the embodiment of FIG. 1A, a flexible 2-axis capacitive goniometer 105 (e.g., the first end 105a) is secured to the patient's ankle with an ankle strap 104. The second end 105b (e.g., on its extender) of the goniometer 105 fits through an adjustable cutting slot 115 in the tibial cutting fixture 106. The first end 105a and the second end 105b of the goniometer 105 communicate with one another, in some embodiments. The end with the electronics and ankle strap 104 (e.g., the first end 105a) establishes a reference plane (e.g., is connected to a reference structure) for the tibial posterior cutting angle. The second end 105b of the goniometer 105 resides in the cutting slot 115 of the tibial cutting fixture 106, and when the surgeon changes a posterior cutting angle by adjusting the tibial cutting fixture 106, the capacitance (e.g., a measurement phenomenon) of the goniometer 105 changes, in some embodiments. In some embodiments, the change in capacitance is calibrated directly to angle changes, and the angle changes may be displayed on the display 102 or transmitted to a computing device. In some embodiments, the relationship of the change in capacitance to angular changes are linear. Many surgeons choose a posterior cutting angle of about 6 degrees. However, the surgeon can choose to use an angle ranging from about 0 degrees to about 10 degrees. The length L of the goniometer can vary from about 2 mm to about 50 mm, about 5 mm to about 45 mm, about 10 mm to about 30 mm, about 2 mm to about 8 mm, about 2 mm to about 5 mm, or about 4 mm in some embodiments. Accuracy is increased with shorter lengths, in some embodiments. In some embodiments, the goniometer 105 may use a change in resistance, current, or magnetic angle to measure an angle change as a measurement phenomenon.

Although the embodiment of FIGS. 1A and 1B describes the first end 105a as attached to a reference plane or structure and the second end 105b as attached to an instrument, the attachment points may be reversed in some embodiments. For example, the end with the electronics (e.g., the first end 105a) may be attached to an instrument, and the opposing end (e.g., the second end 105b) may be attached to a reference plane or structure, in some embodiments.

The flexible goniometer 105 also establishes changes in the valgus angle (outward from the midline) and varus angle (inward toward the midline), in some embodiments.

In some embodiments, the surgeon may remove the second end 105b of the goniometer 105 and tibial cutting fixture 106 from the tibia after the tibia condyles 116 have been cut and place the second end 105b of the goniometer 105 on the condyle cut surface 117 to further check for the correct tibia posterior angle.

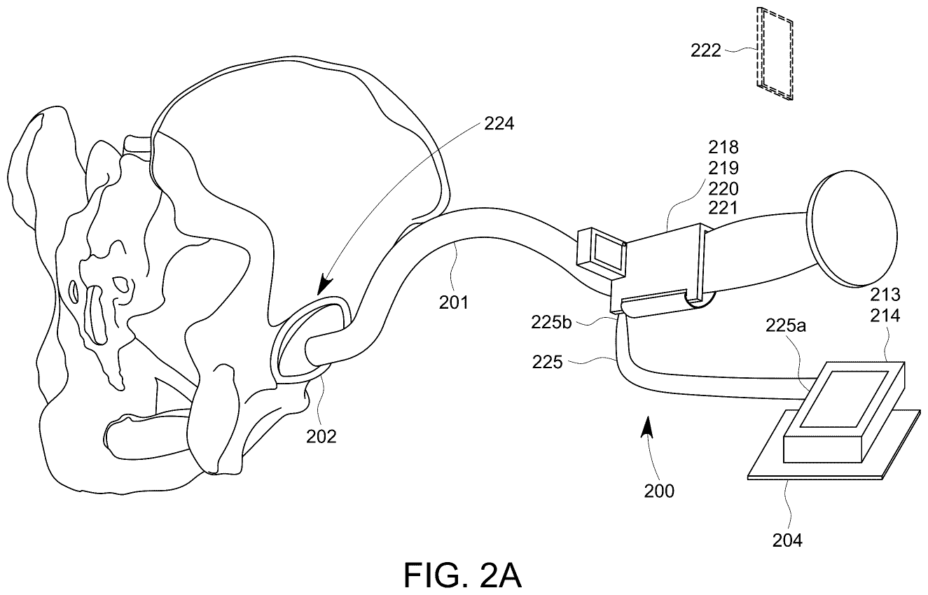

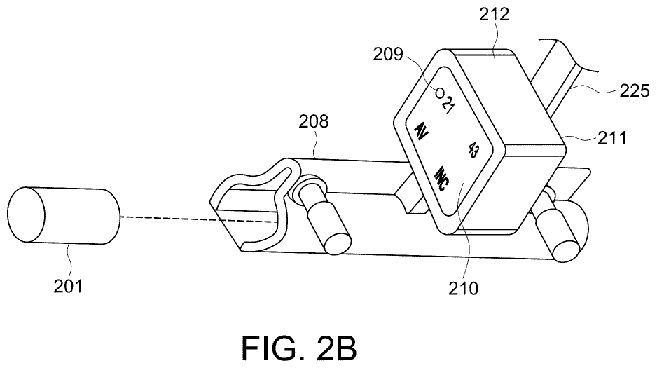

As shown in FIGS. 2A and 2B, an embodiment of a hip arthroplasty measurement system 200 is shown connected on one end to an acetabular handle 201 during Total Hip Arthroplasty (THA). The acetabular handle 201 holds an acetabular cup 202 for installation in the patient's pelvis acetabulum 224 or cotyloid cavity during THA. The electronics side (e.g., first end 225a) of the flexible 2-axis goniometer 225 is connected, in some embodiments, to a reference plane or structure (e.g., the coronal plane 204, the sagittal plane 222, etc.). The second end 225b of the goniometer 225 may be connected to the acetabular handle 201. As described herein, the goniometer 225 provides angular information that establishes the correct orientation of an acetabular cup 202 when installed in the pelvic acetabulum 224. Angles measured may include, but are not limited to, inclination and anteversion, as shown in FIG. 2A.

In FIG. 2B, an embodiment of the components for the measurement system 200 is shown. These include, but are not limited to, a power source, an acetabular handle connecting feature 208, an on-off tare button 209, a display 210 (e.g., LCD or LED), a printed circuit board 211, an electronic housing 212, and a connective means 213 to the coronal plane 204 as a reference plane or structure, in some embodiments. This connective means 213 may include, but is not limited to, an adhesive pad 214 that can be placed on a patient's pelvis, a Mayo stand, or a gurney rail L bracket. In some embodiments, the connective means can also be a snap feature and a clamp screw. The length of the goniometer 225 may be about 2 mm to about 75 mm, about 4 mm to about 60 mm, about 10 mm to about 50 mm, about 15 mm to about 50 mm, or about 15 mm to about 30 mm, in some embodiments.

The acetabular handle connecting feature 208 may take several forms in various embodiments. These include, but are not limited to, a snap feature 218, a tubing clamp 219, a clamp screw 220, and a pinch clamp 221.

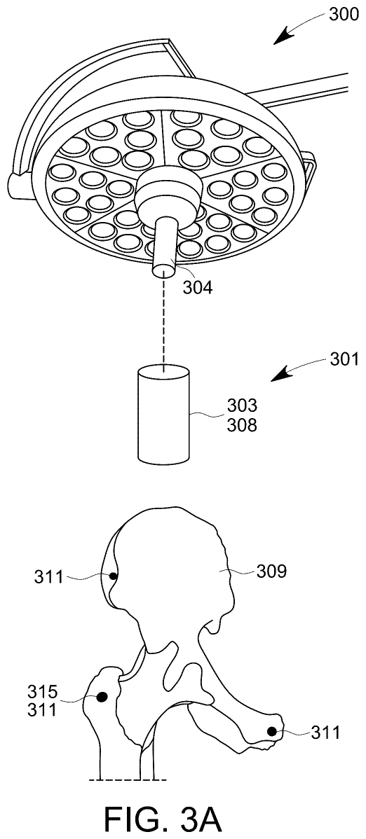

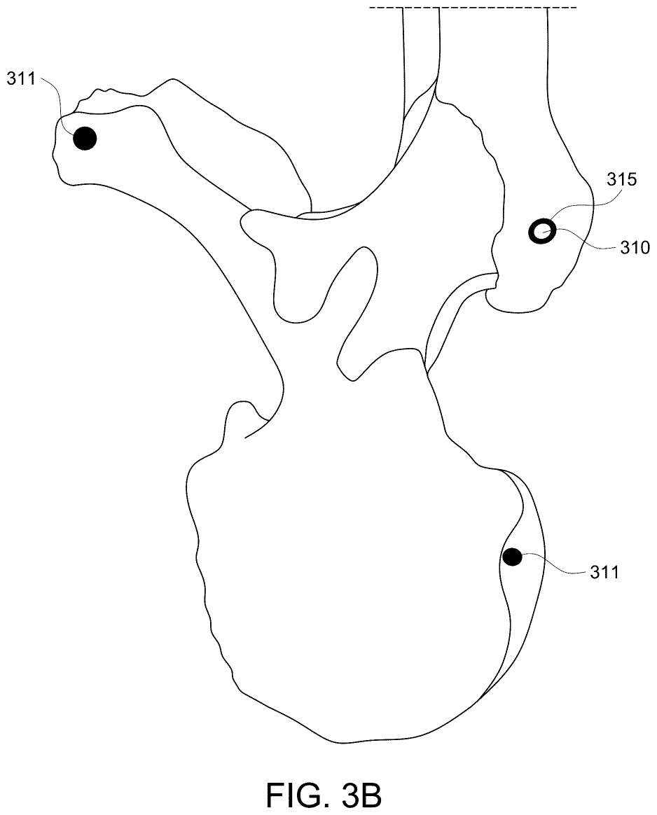

FIG. 3A shows a measurement system 300 that employs a form of advanced digital image processing to measure pelvic leg length and offset, in some embodiments, as a distance measurer 301. These parameters are important in assuring better outcomes for hip arthroplasty by matching the native patient anatomical parameters. In at least this way, better stability and balance can be achieved, in some embodiments. In some embodiments, the advanced digital image processing is a software package employed in astronomical image processing.

The distance measurer 301 includes, in some embodiments, a camera 303 that can be connected to the surgical light handle 304 or to a bracket connected to the gurney rail or to an IV stand. In some embodiments, such a connection to another object may eliminate motion when an image is taken by the camera 303. The system 300 may also include an ultrasonic range finder 308, in some embodiments, to establish a suitable depth of field to assist an autofocus function of the camera 303. Other means for depth of field determination may include, but are not limited to, the use of a set height of a bracket or a set height on an IV stand, in some embodiments. The surgeon or other clinician (without an ultrasonic range finder 308 or other range finder) can just watch a monitor and establish when the pelvic bone features 309 come into clear view when the surgical light handle 304 attached camera 303 is positioned perpendicular to the pelvic bone features 309.

The digital imaging software of the distance measurer 301 works by first establishing a centroid center 310 around at least two markers 311 (e.g., measuring points or dots), in some embodiments, which are placed on anatomical features to be measured. In some embodiments, the markers 311 may be in the form of a bone screw with lid and a target on top, methylene blue dot, or adhesive tape dot. A leg length marker 315 (e.g., a marker 311 on a femur of a patient) will not necessarily be in the same horizontal plane as the other markers 311 because the leg length anatomical feature is inferior to the other anatomical features. However, in some embodiments, a bone screw with a heightened lid and target may be used so that all three markers 311 can be roughly in the same plane, thereby improving image focus and measurement accuracy. In some embodiments, the leg length marker 315 is within about 0.25 mm to about 0.75 mm of the plane of the other markers 311. In some embodiments, the leg length marker 315 is within about 0.3 mm to about 0.7 mm of the plane of the other markers 311. In some embodiments, the leg length marker 315 is about 0.5 mm of the plane of the other markers 311. Depending on surgeon preference, sub-millimeter accuracy may not be used. In some embodiments, the centroid center 310 may still be established with the digital imaging software when the leg length marker 315 is slightly out of focus.

To determine the centroid center 310 of a marker 311 in an image, a processor in communication with the camera 303 may first identify the pixel coordinates of a perimeter of a marker 311 (e.g., by counting pixels having sufficient, predetermined contrast to their surroundings) in an image. Using the pixel coordinates of the perimeter of the marker 311, the processor may then calculate at least an approximate center or centroid center 310 of the marker within the image.

Once the centroid centers 310 of at least two markers 311 are determined in a first image taken by the camera 303, the processor employing digital imaging software may count pixels of the first image between the at least two centroid centers 310 of the markers 311. A measurement in millimeters is determined by taking the counted pixels between the two target centers and then multiplying by the pixel width. More advanced cameras with 8 megapixel and 4K resolution may be employed in some embodiments. With 4K cameras, there is 4000 horizontal pixel resolution, so accuracy is improved. However, cameras with 3-to-12-megapixel resolution can also be utilized as well. The distance may be displayed on a display in communication with the processor, in some embodiments.

For example, by counting the number of pixels between a centroid center 310 of a leg length marker 315 and a centroid center 310 of a marker 311 on a pelvis, the distance measurer 301 may determine a length between a current placement of a femur relative to the pelvis during an operation and output or display that length. As a user of the system 300 moves the femur or pelvis during an operation, the distance measurer 301 may capture a second image, calculate the distance again, and update a display with the new distance value. In some embodiments, the distance measurer 301 may be used in combination with a goniometer (e.g., goniometer 105 of FIGS. 1A-1B, goniometer 225 of FIGS. 2A-2B, etc.) in a measurement system (e.g., system 100 of FIGS. 1A-1B, system 200 of FIGS. 2A-2B, etc.) to provide both distance measurements and angle measurements to a user during an operation.

FIG. 3B shows an example of a pelvic bone with markers 311 (e.g., a bone screw with a lid and target). At least one marker 311 on a femur may be a leg length marker 315, in some embodiments. Centroid centers 310 may be calculated by a processor for at least two markers 311 in an image captured by the camera 303.

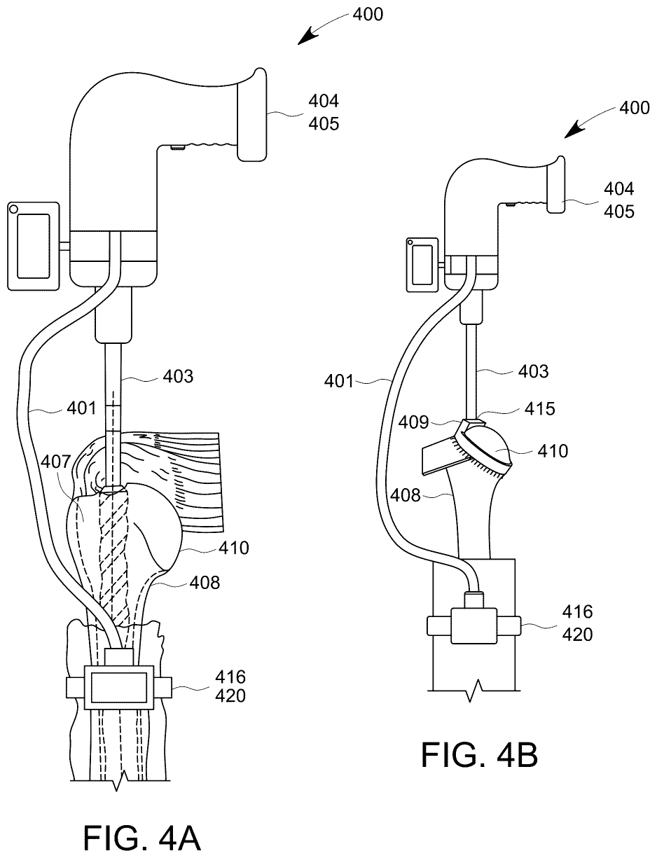

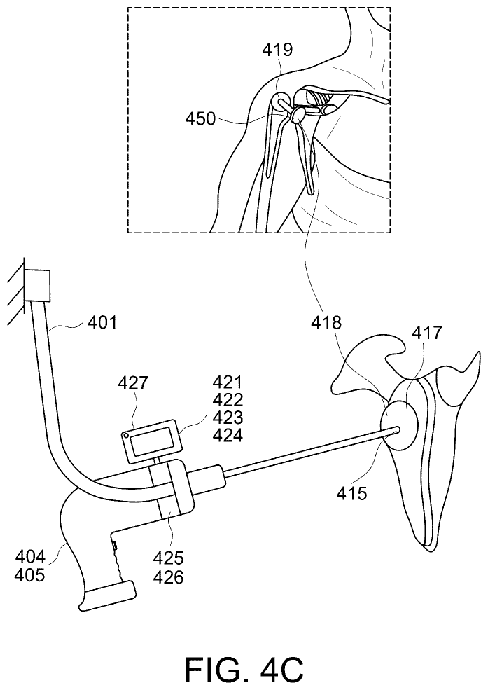

FIGS. 4A, 4B, and 4C illustrate an embodiment of a measurement system 400 for reverse and standard total shoulder arthroplasty employing a flexible goniometer 401. The flexible 2-axis goniometer 401 is shown connected on one side (e.g., a second end) to a pilot hole drill 403 that is powered by a hand drill 404 or a power drill 405. The other side (e.g., a first end) of the flexible 2-axis goniometer 401 (with electronics) is a parallel connection to the longitudinal axis of the humerus (e.g., a reference plane or structure). This connection can be made with an adhesive pad 416 or a strap 420 around the arm, in some embodiments. The drills 403, 404, or 405 are used, in some embodiments, to establish a starting point for cutting the humeral head 410 and broaching the humerus cancellous bone 407. The pilot drill 403 may be in line with the longitudinal axis of the humerus 408 for broaching the humerus cancellous bone 407, in some embodiments.

As shown in the embodiment of FIG. 4B, the goniometer 401 serves as a way to establish that the pilot hole drill 403 is either in line with the longitudinal axis of the humerus 408 or at a correct angle for installing the humeral head cutting fixture 409 by providing correct guide angles. Further, the correct guide angles establish a guide for the cutting fixture 409 to be fitted correctly on the humeral head 410, in some embodiments. Once the correct cutting fixture installation angles are established, the cutting fixture 409 can be used to cut the humeral head 410 at about 135 degrees or about 155 degrees. In some embodiments, the cutting fixture 409 can be used to cut the humeral head 410 at about 120 degrees to about 150 degrees, about 125 degrees to about 145 degrees, about 130 degrees to about 140 degrees, about 140 degrees to about 170 degrees, about 145 degrees to about 165 degrees, or about 150 degrees to about 160 degrees. The humeral head pilot hole drill position 415 in the x-y plane is not as critical, in some embodiments. The pilot hole drill position 415 can be accomplished freehand or with a simple fixture, in some embodiments. However, pilot hole drill starting angles are important, in some embodiments. The simplicity of the measurement system 400, in some embodiments, can eliminate navigation computer and robot complexity.

Similarly, in FIG. 4C the scapula glenoid 417 also requires a pilot hole drill 403 to establish the correct angle for a base plate 418 and ball head 419 for reverse shoulder arthroplasty or a glenoid cup 450 for standard total shoulder arthroplasty, in some embodiments. In reverse shoulder arthroplasty the base plate glenoid angle is required to be an inferior angle of about 12 degrees to about 20 degrees, in some embodiments. In some embodiments, the inferior angle is about 8 degrees to about 24 degrees, or about 10 degrees to about 22 degrees. By creating an inferior angle, the shoulder deltoid muscles are kept in compression, thus creating stability and balance for the shoulder, in some embodiments. Standard total shoulder arthroplasty the glenoid pilot hole angle needs to be at a slight angle of +/− about 0 degrees to about 4 degrees anteversion or retroversion, in some embodiments. In some embodiments, the slight angle of the glenoid pilot hole angle is about +/−0 degrees to about 8 degrees, or about 0 degrees to about 6 degrees.

In FIG. 4C a plurality of internal components, in some embodiments is shown. On the electronics side (e.g., a first end) of the system 400 these include a battery 421, electronic board 422, display 423, electronic housing 424, pilot drill collar 425, hand/power drill adhesive pad 426 and a tare/on/off button 427. On the pilot drill side connection of the device these include a pilot hole drill collar 425 or an adhesive pad 426 that is adhered to a hand drill 404 or power drill 405. The length of the goniometer 401 can vary from about 2 mm to about 75 mm, about 4 mm to about 65 mm, about 10 mm to about 55 mm, or about 15 mm to about 50 mm.

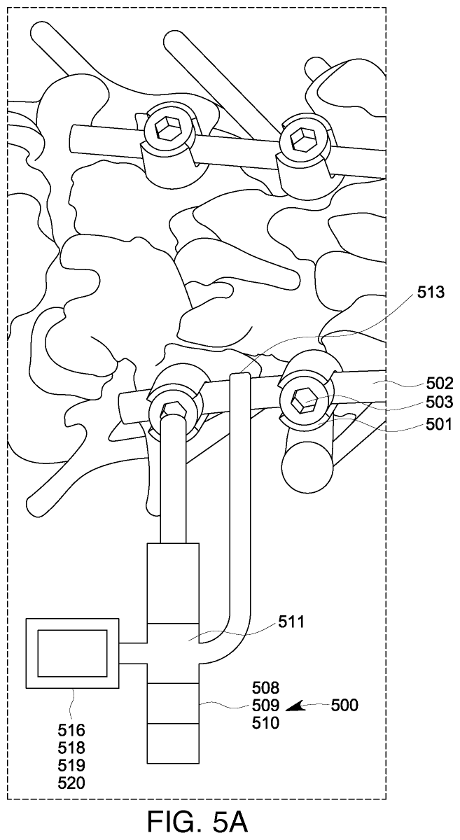

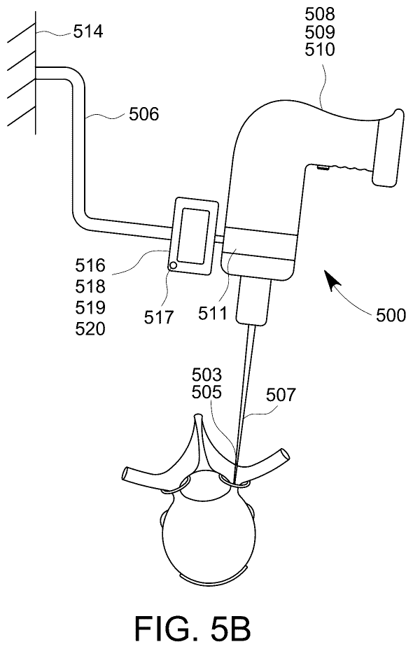

FIGS. 5A and 5B show embodiments of a measurement system 500 for spinal fusion surgery. The scope of use for the embodiment of FIGS. 5A and 5B includes but is not limited to cervical, thoracic and lumbar fusion surgeries. In the embodiment of FIG. 5A the 2-axis flexible goniometer 506 is shown connected to the top or “tulip” of a pedicle screw 501 and a fixation rod 502 with a set screw 503. The system 500 on the electronic side (e.g., a first end) is connected to the top of the tulip of a pedicle screw 501 and the opposite side (e.g., a second end) is connected to the fixation rod 502 with a snap-on collar 513, in some embodiments. Compared to the hip, knee and shoulder the spinal column sees more force. Therefore, maintaining the proper angle between the “tulip” head of pedicle screw 501 and fixation rod 502 is crucial especially without forcing it, in some embodiments. The ideal angle between the fixation rod 502 and tulip top of pedicle screw 501 is about 90 degrees, in some embodiments. The angle needs to be about 90 degrees because a deviation can cause either mechanical stress on the spinal fixation assembly which can lead to loosening or fracturing, in some embodiments. Therefore, surgeons should check the rod/screw tulip angle, in some embodiments, before and after tightening the two together with the measurement system 500 including goniometer 506 as shown.

Some pedicle screws 501 have a “fast” thread or steep helix angle as well as a large thread depth for better fixation in soft cancellous bone, in some embodiments. Fixation in hard cortical bone is sometimes better suited to a fine thread with smaller thread depth and shallower pitch, in some embodiments. Once a pilot hole is completed in the vertebrae pedicle there is a tendency for the pedicle screw 501, regardless of the thread geometry, to drift from the pilot hole 505 axis in some embodiments. It is imperative that the surgeon install pedicle screws 501 that avoid the pedicle neuro-vascular beds, in some embodiments. Therefore, matching the pilot hole 505 angular position in two dimensions to the two-dimensional pedicle screw installation angle is crucial, in some embodiments. The attachment means for the goniometer 525 is a snap-on collar 513 for the fixation rod 502 and a snap-on collar for a hand drill/driver, in some embodiments.

In some cases, spinal surgeons employ compass or protractor like instruments, O arm (CT scan) images, robots and navigation devices to aid in spinal fixation component installation. The National Institute of Health has studied these spinal fixation surgery technologies and found that the outcomes are not any better than by freehand. Therefore, the measurement system 500 (e.g., including the goniometer 506 of FIGS. 5A and 5B) as described, herein, provide a more accurate and safe installation of spinal fixation components.

In FIG. 5B, a plurality of components is shown for the measurement system 500, in some embodiments. On one end of the goniometer 506 (e.g., a second end) is a probe 507 with a Phillips or straight slot head that can either fit into a pilot hole 505 or the pedicle set screw 503. The probe 507 can be connected to a hand drill/driver 508, power drill/driver 509 or a screwdriver 510 with a snap-on collar 511.

Opposite the electronic side of the goniometer 506 (e.g., a first end) is an attachment means to a reference plane 514 in some embodiments. The reference plane for the pilot hole 505 and pedicle screw 501 can be sagittal or coronal in some embodiments. The relative angle between pilot hole 505 and pedicle screw 501 is the important parameter, not necessarily the absolute angle, in some embodiments. The electronic side (e.g., second end) attachment means can be brackets, adhesive pads and snap-on features.

The componentry may be the same as embodiments of the orthopedic devices described herein. These include a battery 516, connecting features as described previously, an-off/tare button 517, an LCD or LED display 518, a PC Board 519 and an electronic housing 520.

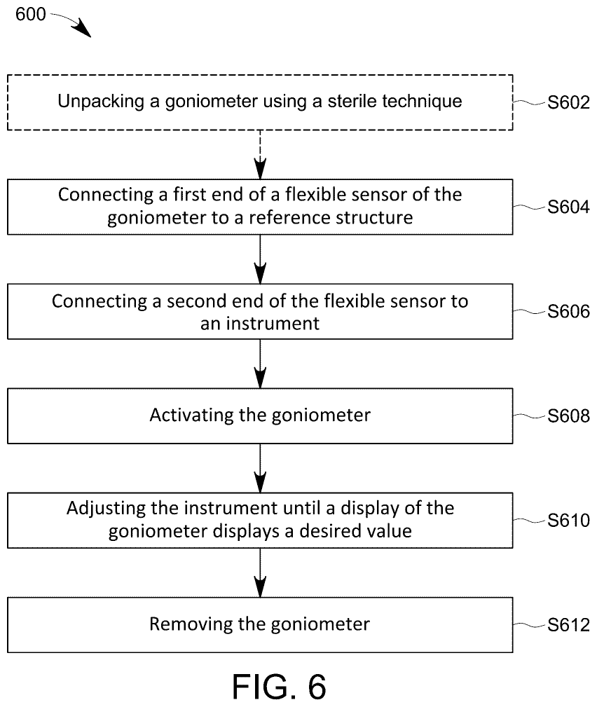

In FIG. 6 is a block diagram for an embodiment of a method 600 for the general goniometer installation and operational flow. In some embodiments, the method includes optionally unpacking a goniometer using sterile technique in block S602, connecting a first end of a flexible sensor of the goniometer to a reference structure in block S604, connected a second end of the flexible sensor to an instrument in block S606, activating the goniometer in block S608, adjusting the instrument until a display of the goniometer displays a desired value in block S610, and removing the goniometer in block S612. In some embodiments, the goniometer is the goniometer of any of FIGS. 1A-1B, 2A-2B, 4A-4C, or 5A-5B or any other embodiment as described herein.

In some embodiments, the method 600 includes optionally unpacking a goniometer using sterile technique in block S602. For example, the goniometer may arrive in an operating room packaged in sterile packaging to prevent the transmission of disease vectors from a manufacturer of the goniometer to the user and patient and therefore may be unpackaged according to sterile practices.

In some embodiments, the method 600 includes connecting a first end of a flexible sensor of the goniometer to a reference structure in block S604. In some embodiments, the reference structure is a patient's limb or body part to be operated on or near a location of operation, such as an arm, leg, or pelvis. In some embodiments, the reference structure is a Mayo stand, a gurney rail L bracket, or another fixed component of an operating room. In some embodiments, the first end connected to the reference structure may be aligned with a reference plane of the patient such as the coronal plane and the sagittal plane. In some embodiments, the first end may be connected by, for example, a snap feature, a clamp screw, an adhesive pad, or a strap.

In some embodiments, the method 600 includes connecting a second end of the flexible sensor to an instrument in block S606. In some embodiments, the instrument may be, but is not limited to, a bone cutting fixture, an acetabular handle, a drill, or a fixation rod. In some embodiments, the second end may be connected to the instrument by, for example, an adhesive, a snap collar, or another fitting.

In some embodiments, the method 600 includes activating the goniometer in block S608. For example, a user may press a power button to power on the goniometer. In some embodiments, the power button automatically tares the device on powering on. In some embodiments, the goniometer comprises a separate tare button from a power switch.

In some embodiments, the method 600 includes adjusting the instrument until a display of the goniometer displays a desired value, or until the goniometer outputs a desired value, in block S610. For example, an operator may adjust the position of a bone cutting fixture, acetabular handle, etc. as part of an operation. As described herein, the adjustment of the instrument moves the second end of the flexible sensor relative to the first. This change in relative position may cause a change in electrical capacitance, resistance, piezoelectric voltage or magnetics in the device, which may be correlated to an angle measurement in some embodiments. The goniometer may display a value for an angle measurement on a display.

In some embodiments, the method 600 includes removing the goniometer in block S612. In some embodiments, the first and second end of the flexible sensor are disconnected from the reference structure and instrument, respectively. In some embodiments, the goniometer is disposable. In some embodiments, the goniometer may be sterilized and used in a subsequent operation.

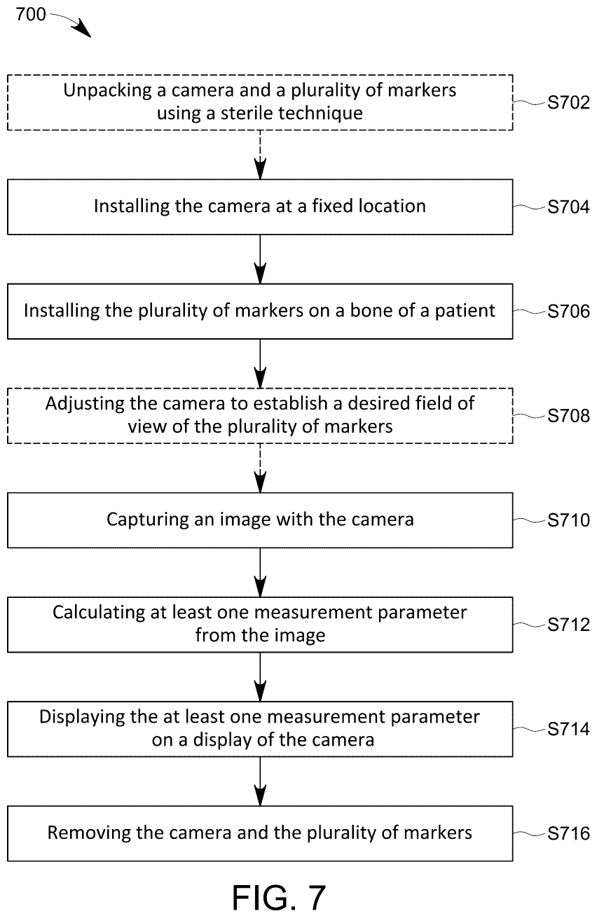

In FIG. 7 is a block diagram for an embodiment of a method 700 for the installation and operational flow of a distance measurer as part of a measurement system. In the embodiment of FIG. 7, the distance measurer is employed in a hip arthroplasty for determining offset and leg length but may employed in, or modified for, other surgical operations in other embodiments . . . . In some embodiments, the method includes unpacking a camera and a plurality of markers using a sterile technique in block S702, installing the camera at a fixed location in block S704, installing the markers on a bone of a patient in block S706, optionally adjusting the camera to establish a desired field of view of the plurality of markers in block S708, capturing an image with the camera in block S710, calculating at least one measurement parameter from the image in block S712, displaying the at least one measurement parameter on a display of the camera in block S714, and removing the camera and markers in block S716.

In some embodiments, the method 700 optionally includes unpacking a camera and a plurality of markers using a sterile technique in block S702. For example, the camera and the plurality of markers may arrive in an operating room packaged in sterile packaging to prevent the transmission disease vectors from a manufacturer to the user and patient and therefore may be unpackaged according to sterile practices.

In some embodiments, the method 700 includes installing the camera at a fixed location in block S704. For example, the camera may be installed on a surgical light handle, a bracket connected to the gurney rail, or to an IV stand. In some embodiments, the camera is positioned such that it has a view over the area of a patient to be operated on.

In some embodiments, the method 700 includes installing the plurality of markers on a bone of a patient in block S706. In some embodiments, the markers may be bone screws with lid and a target on top, methylene blue dots, adhesive tape dots, or a combination thereof. In some embodiments, the markers may be installed on key features of anatomy for an operation.

In some embodiments, the method 700 optionally includes adjusting the camera to establish a desired field of view of the plurality of markers. For example, the camera may include an ultrasonic range finder to assist with an autofocus function of the camera . . . . In some embodiments, the desired field of view includes at least two of the plurality of markers in focus. In some embodiments, the desired field of view includes each of the plurality of markers in focus.

In some embodiments, the method 700 includes capturing an image with the camera in block S710. In some embodiments, the image includes at least two of the plurality of markers. In some embodiments, the image includes each of the plurality of markers.

In some embodiments, the method 700 includes calculating at least one measurement parameter from the image in block S712. For example, a processor in communication with the camera and a memory storing machine-readable instructions may determine a centroid center for at least one marker in the image, in some embodiments. In some embodiments, the processor may determine a centroid center for at least two markers in the image. As described herein, the processor may determine a centroid center by detecting a perimeter of a marker in an image by detecting predetermined differences in contrast between the marker and their surroundings, in some embodiments. From the perimeter in pixels, the processor may calculate at least an approximate centroid center for a marker. The processor may in some embodiments count pixels of the image between a first and second centroid center to form a pixel path length. In some embodiments, the pixel path length is along the shortest path between the first and second centroid center. The processor may multiply a pixel count by a pixel width to determine a measurement parameter for the image, in some embodiments. In some embodiments, the measurement parameter may include a distance.

In some embodiments, the method 700 includes displaying the at least one measurement parameter on a display of the camera in block S714. For example, the camera may display a distance value associated with at least one of the plurality of markers on a display.

In some embodiments, the method 700 includes removing the camera and the plurality of markers in block S612. In some embodiments, the camera and the plurality of markers are disposable. In some embodiments, the camera and the plurality of markers may be sterilized and used in a subsequent operation.

The systems and methods of some embodiments may be embodied and/or implemented at least in part as a machine configured to receive a computer-readable medium storing computer-readable instructions. The instructions are preferably executed by computer-executable components preferably integrated with the system and one or more portions of the processor on the goniometer and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “strap” may include, and is contemplated to include, a plurality of straps. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A measurement system for orthopedic surgery, comprising:
a goniometer comprising a sensor comprising a first end opposite a second end,
wherein the first end is coupled to a reference structure, wherein the sensor is configured to a generate measurement phenomenon correlated to a relative position of the first end and the second end, and wherein the measurement phenomenon is selected from a group consisting of: an electrical resistance, a capacitance, a current, a pressure on a piezoelectric substrate, a magnetic field, a magnetic angle, or a mechanical force;
a processing unit communicatively coupled to the goniometer; wherein the processing unit comprises a processor and a memory storing machine-readable instructions that when executed by the processor, cause the processor to perform a method comprising:
receiving a first signal indicative of a first value for the measurement phenomenon;
correlating the first signal with a first angle between the first end and the second end of the sensor; and
outputting a value of the first angle to a display in communication with the processing unit.

2. The measurement system of claim 1, further comprising:
a distance measurer comprising a camera communicatively coupled to the processing unit, wherein the machine-readable instructions when executed by the processor further cause the processor to perform a method comprising:
capturing a first image, wherein the first image includes at least a first marker and a second marker;
identifying, in the first image, at least a first centroid center and a second centroid center of the first and second markers, respectively;
determining a measurement parameter based on the identified first centroid center and a second centroid center of the first and second markers, respectively; and
outputting the measurement parameter,
wherein the camera is located at a fixed position during an operation of the measurement system.

3. The measurement system of claim 2, wherein the determining a measurement parameter further comprises:
counting pixels between the first and second centroid centers along a shortest path between the first and second centroid centers to calculate a first pixel path length; and
multiplying the first pixel path length by a pixel width to calculate a first distance path length as a measurement parameter.

4. The measurement system of claim 2, wherein the distance measurer further comprises an ultrasonic range finder.

5. The measurement system of claim 1, wherein the second end of the sensor is coupled to a surgical instrument.

6. The measurement system of claim 5, wherein the surgical instrument is selected from the group consisting of: a bone cutting fixture, an acetabular handle, and a drill.

7. The measurement system of claim 1, further comprising:
an elongate member between the first end and the second end.

8. The measurement system of claim 7, wherein the elongate member includes an adjustable length.

9. The measurement system of claim 1, further comprising:
another sensor configured to generate additional measurement phenomenon correlated to a relative position of the first end and the second end in a second angle between the first end and the second end of the another sensor.

10. The measurement system of claim 9, wherein the method further comprises:
receiving a second signal indicative of a second value for the additional measurement phenomenon;
correlating the second signal with the second angle between the first end and the second end of the another sensor; and
displaying a value of the second angle on the display in communication with the processing unit.

11. The measurement system of claim 9, wherein the additional measurement phenomena phenomenon is selected from a group consisting of: an electrical resistance, a capacitance, a current, a pressure on a piezoelectric substrate, a magnetic field, a magnetic angle, or a mechanical force.

12. The measurement system of claim 1, wherein the first angle comprises information that is selected from a group consisting of: a tibial posterior angle, a varus, a valgus, an inclination, an anteversion, a reverse and total shoulder angle, or a humerus cut angle.

13. The measurement system of claim 1, wherein the goniometer comprises a cutting slot to provide correct guide angles for cutting during the orthopedic surgery.

14. A measurement system for orthopedic surgery, comprising:
a goniometer comprising a first sensor and a sensor having a first end opposite a second end,
wherein the first end is coupled to a reference structure, wherein the first sensor is configured to a generate measurement phenomenon correlated to a relative position of the first end and the second end;
a processing unit communicatively coupled to the goniometer; wherein the processing unit comprises a processor and a memory storing machine-readable instructions that when executed by the processor, cause the processor to perform a method comprising:
receiving a first signal indicative of a first value for the measurement phenomenon;
receiving a second signal indicative of a second value for an additional measurement phenomenon;
correlating the first signal with a first angle between the first end and the second end of the first sensor;
correlating the second signal with the second angle between the first end and the second end of the second sensor; and
outputting a value of the first angle and a value of the second angle to a display in communication with the processing unit.

15. The measurement system of claim 14, wherein the second end of the sensor is coupled to a surgical instrument.

16. The measurement system of claim 15, wherein the surgical instrument is selected from the group consisting of: a bone cutting fixture, an acetabular handle, and a drill.

17. The measurement system of claim 14, further comprising:
an elongate member between the first end and the second end.

18. The measurement system of claim 17, wherein the elongate member includes an adjustable length.

19. The measurement system of claim 14, wherein the additional measurement phenomena phenomenon is selected from a group consisting of: an electrical resistance, a capacitance, a current, a pressure on a piezoelectric substrate, a magnetic field, a magnetic angle, or a mechanical force.

20. The measurement system of claim 14, wherein the first angle comprises information that is selected from a group consisting of: a tibial posterior angle, a varus, a valgus, an inclination, an anteversion, a reverse and total shoulder angle, or a humerus cut angle.
